(19) United States
(12) Patent Application Publication
(10) Pub. No.: US 2018/0224430 A1
(43) Pub. Date: Aug. 9, 2018

(54) Method of Estimating the Incidence of In Vivo Reactions to Chemical or Biological Agents Using In Vitro Experiments

(71) Applicant: Coyne Scientific, LLC, Atlanta, GA (US)

(72) Inventors: Kevin P. Coyne, Atlanta, GA (US); Shawn T. Coyne, Smyrna, GA (US); Anh Nguyen, Ph.D., Mableton, GA (US)

(21) Appl. No.: 15/749,499

(22) PCT Filed: Aug. 18, 2016
(86) PCT No.: PCT/US16/47540

§ 371 Date: Jan. 31, 2018

Related U.S. Application Data
(60) Provisional application No. 62/206,664, filed on Aug. 18, 2015.

(51) Int. Cl. G01N 33/50; G06F 19/24; G16H 50/70

(52) U.S. Cl. CPC G01N 33/5014; G01N 2800/52; G16H 50/70; G06F 19/24

(57) ABSTRACT

A method is provided for estimating or predicting, a priori, the incidence or degree of biological reaction by an individual in a population of interest when exposed to a chemical or biological agent of interest in vivo. The method uses a combination of data from in vitro experiments using the agent of interest, from in vitro experiments using a reference agent, and data obtained regarding the incidence or degree of the in vivo effects of the reference agent.

### FIELD OF THE INVENTION

The present application relates to the field of analysis of biological reactions such as pharmaceutical toxicology, industrial chemical toxicology, pharmaceutical efficacy and the study of infectious diseases.

### BACKGROUND

Individual members of a mammalian population often have biologically different reactions from one another when experiencing an identical exposure to an identical chemical or biological agent. These different reactions result largely from differences in the genetic makeup of the individuals. Differences in genetic makeup sometimes result in only a portion of the population reacting, beneficially or adversely, to such an exposure.

Limitations in the ability to estimate the incidence of beneficial or adverse biological reactions in a mammalian population to chemical or biological agents, without actually exposing members of that population to the chemical or biological agents, can result in costs that are extremely high to society.

In the field of pharmaceutical toxicology, the discovery of toxicity reactions in some but not all members of a population is a major cause of attrition throughout each stage of the drug development process, including even the later stages of the development process. Late stage attrition (i.e., during clinical trials, or post-approval) is particularly costly for pharmaceutical companies because the failure to detect toxicity in candidate compounds earlier in the development process causes “wasted” investment of time, effort, and millions of dollars in certain compounds before their toxicity is discovered.

Similarly, in the field of industrial chemical toxicology, discovery of toxicity reactions by a portion of the population via in vivo exposure can be costly to industrial chemical companies, should such toxicities lead to the prohibition of sale of a profitable product, costly clean-up of environmental damage, or financial compensation to persons harmed by a toxic chemical.

Separately, in the field of pharmaceutical efficacy, delays during the drug development process in distinguishing between candidate compounds that will be widely effective among a target patient population, versus those that will benefit only a narrow subset of a target patient population, can also cause “wasted” investments in compounds that are later found to be ineffective in most patients. However, early stage in vivo testing of candidate compounds is often not feasible for ethical reasons.

In the study of infectious diseases, a wide variety of diseases (e.g., Avian Flu, Ebola, etc.) exist for which potential rates, or incidence, of infection among humans are of great importance to public health. However, these rates of infection cannot be tested in vivo for obvious ethical reasons.

For all of the above-described reasons and additional reasons, the development of a method for estimating the incidence, in a population, of beneficial or adverse biological reactions to chemical or biological agents early and relatively inexpensively, based on in vitro experiments, would be quite valuable in a wide variety of circumstances.

As part of this development, the establishment of an endpoint score on an in vitro experiment that is indicative of a threshold level of a beneficial or adverse biological reaction would be useful.

The present invention provides methods for accomplishing these objectives, by combining the results of in vitro tests with certain data available from a previous in vivo experience.

Of note, for purposes of clarity and brevity, examples cited in the remainder of this application will draw mainly from one of the above-described fields—the testing of pharmaceuticals for toxicity. However, the other above-described fields involve similar or analogous principles, and all instances, not limited to those above, involving estimating or predicting, a priori, the potential incidence of biological reactions (or the incidence of biological reactions above or below a specified threshold) by individuals in a population of interest when exposed to a chemical or biological agent of interest in vivo by using a combination of in vitro experiments on the agent of interest and reference agent and data obtained regarding the incidence of the in vivo effects of that reference agent are included within the scope of the method provided herein, as are all instances of using in vivo data to establish the level for an endpoint score for an in vitro experiment that is indicative of a threshold level of impact of biological reactions.

In recent years, researchers have attempted to develop methods to predict various aspects of toxicity earlier in the drug development process by applying candidate compounds to cells in vitro, where the cells are derived from various types of stem cells. To date, these avenues of inquiry have been focused on four objectives—none of which involve estimating or predicting the in vivo incidence of toxicity reactions (or the incidence of toxicity reactions above a specified level) in a population of interest, which is the domain of the method provided herein.

The first objective of toxicity studies to date is to examine, in depth, the impact of a compound on cells from a single human donor. While these in vitro experiments may compare the relative severity of cellular-level reactions of a single donor's cells to different compounds or differing doses of a single compound, the severity of reaction in a single individual is not predictive of the portion of a population that will suffer a reaction (or a reaction above or below a specified threshold). By definition, measures of relative severity of a single individual offer little assistance to answering the incidence question.

The second objective of toxicity studies to date is to develop methodologies to translate between (1) the dose and administration method of a compound delivered to a living patient in vivo, and (2) the concentration of that same compound administered to the cells or tissues in vitro that represents the “same dose.” These methodologies are often referred to as IVIVE, or In Vitro-In Vivo Extrapolation, methodologies. While certain embodiments of the method provided herein may make use of IVIVE methodologies at certain points in the processes, IVIVE methodologies alone are outside the scope of the present method. Recent examples of IVIVE methodologies include: Yoon, M., Campbell, J. L., Andersen, M. E., & Clewell, H. J. [2012], “Extrapolation of Cell-Based Toxicity Assay Results’, Critical Reviews in Toxicology, 42(8), 633-652; Quignot, N., Hamon, J., Bois, F. Y. [2014], “Extrapolating In Vitro Results to Predict Human Toxicity”, In Vitro Toxicology Systems, Methods in Pharmacology and Toxicology, VIII, 531-550; and Quignot, Nadia; Bois, Frédéric Yves [2013], “A Computational Model to Predict Rat Ovarian Steroid Secretion from In Vitro Experiments with Endocrine Disruptors”, PLoS ONE, 8 (1), e53891.

The third objective of toxicity studies to date is to develop a mechanistic understanding of the pharmacokinetic processes (known in the field as Absorption, Distribution, Metabolism and Excretion processes or ADME) that take place when a compound is administered in vivo, including how those processes may alter the concentration or composition of a compound before, during, and potentially after, the compound affects the cells, tissues, or organs under study, and how to model those changes when attempting to translate in vitro results to likely in vivo effects. To-date, efforts toward this objective have been confined to analyses conducted at the level of the human species as a whole—the work has not progressed to elucidating the differences involved in modeling one human being versus another, which is a pre-requisite for providing insights on the incidence of effects. As with the second objective described above, while certain embodiments of the method provided herein may involve these methodologies at certain points in their processes, these methodologies alone are outside scope of the present methods. Recent examples of these methodologies include: Louisse, J., de Jong, E., van de Sandt, J. J. M., Blaauboer, B. J., Woutersen, R. A., Piersma, A. H., et al [2010], “The Use of In Vitro Toxicity Data and Physiologically Based Kinetic Modeling to Predict Dose-Response Curves for In Vivo Developmental Toxicity of Glycol Ethers in Rat and Man”, Toxicological Sciences, 118(2), 470-484; Sung, J. H.; Esch, M. B.; Shuler, M. L. [2010], “Integration of In Silico and In Vitro Platforms for Pharmacokinetic-Pharmacodynamic Modeling”, Expert Opinions in Drug Metabolism and Toxicology, 6, 1063-1081; and Blaauboer, B. J. [2010], “Biokinetic Modeling and In Vitro In Vivo Extrapolations”, Journal of Toxicology and Environmental Health, Part B, 13:242-252.

The fourth objective of toxicity studies to date, which has only recently begun to be pursued, is to correlate in vitro data on a compound with the most elemental binary fact about in vivo toxicity—i.e., whether or not there has been any report of a toxic reaction in previous clinical trials or post-approval usage (see Pointon, A., Harmer, A. R., Dale, I. L., Abi-Gerges, N., Bowes, J., Pollard, C., & Garside, H. [2014], “Assessment of Cardiomyocyte Contraction in Human-Induced Pluripotent Stem Cell-Derived Cardiomyocytes”, Toxicological Sciences, 144(2), 227-237; and Doherty, K. R., Talbert, D. R., Trusk, P. B., Moran, D. M., Shell, S. A., & Bacus, S. [2015], “Structural and Functional Screening in Human Induced-Pluripotent Stem Cell-Derived Cardiomyocytes Accurately Identifies Cardiotoxicity of Multiple Drug Types”, Toxicology and Applied Pharmacology, 285(1), 51-60). Importantly, work to-date has not progressed to the point of attempting an estimate or prediction on a novel compound, nor does it speak to the question of incidence in a population.

A number of methods for utilizing cells derived from large samples of donors to address the incidence issue have been previously described (See, e.g., Coyne et al., PCT Application No. PCT/US14/45499, entitled “Methods for Predicting Responses to Chemical or Biologic Substances”; Coyne et al., PCT Application No. PCT/US14/53819, entitled “Methods for Genetically Diversified Stimulus-Response Based Gene Association Studies”). Those references involve a variety of methods by which a researcher can estimate the likely incidence of toxicity reactions to a compound of interest, but they are distinguished from the method provided herein in that they do not directly utilize in vivo data, which may prove more reliable in projecting in vivo responses.

What is needed is an improved method for utilizing in vitro assays to estimate or predict, a priori, the potential incidence of biological reactions by individuals in a population of interest when exposed to a novel compound of interest in vivo. As a distinct step in that process, an improved method is needed for using in vivo data about the incidence of effects of particular exposures to an agent in vivo as well as in vitro to establish the level for an endpoint score for an in vitro experiment that is indicative of a threshold level of impact of biological reactions in vivo.

The method described herein meets this need by using a combination of in vitro experiments on a compound of interest and a reference compound and data obtained regarding the incidence of the in vivo effects of the reference compound.

### SUMMARY OF THE INVENTION

Provided herein is a method of determining the potential incidence or degree of a beneficial or adverse biological reaction by an individual in a population of interest when exposed in vivo to an agent of interest. Preferably, the population of interest is a mammalian population, preferably the individual is a human and the agent of interest is a chemical or biological agent of interest. The method includes combining results of in vitro exposure to the agent of interest, results of in vitro exposure to a reference agent, and data obtained regarding the incidence of the in vivo effects upon exposure to the reference agent. This combination is utilized to either establish one or more scores for one or more endpoints of an in vitro experiment that can serve as a proxy for estimating a threshold level of a beneficial or detrimental effect, or for estimating or predicting, a priori, the potential incidence of a beneficial or adverse biological reaction by an individual in a population of interest when exposed in vivo to an agent of interest.

In accordance with the method, the incidence or degree of the biological reaction in a sample of the population of interest is determined, wherein the sample includes cells or tissues from at least 10 individual donors of the population of interest, or an analogous population, when exposed to at least one reference agent in vivo; the incidence or degree of the biological reaction in a sample of the population of interest is determined, wherein the sample includes cells or tissues from at least 10 individual donors of the population of interest, or an analogous population, when exposed to at least one reference agent in vitro; the incidence or degree of the biological reaction of individuals in the population of interest when exposed to the chemical or biological agent of interest in a least one in vitro assay is determined; and the incidence or degree of the biological reaction by an individual in the population of interest based on the incidence or degree of the biological reaction when exposed to at least one reference agent in vivo is estimated or predicted. The individual of the population of interest is a mammal.

Also provided is a method for establishing a threshold endpoint score for an in vitro assay to produce an estimate of the incidence in a population of interest of a related effect in vivo, by:
(a) selecting one or more in vitro tests for which an endpoint measured by those tests may exhibit a mathematical transform with the incidence of the biological reaction in vivo;
(b) identifying at least 10 individual donors as a sample of the population of interest, and obtaining separately for each donor, a cell, tissue, organ, aggregates thereof or cells, tissues, or organs derived therefrom for the selected in vitro test;
(c) administering the selected in vitro test on each donor of each sample by exposure to a reference chemical or biological agent, wherein the incidence of the biological reaction has been determined for the in vivo exposure of the sample population of interest or an analogous population to the reference chemical or biological agent; wherein the reference chemical or biological agent and the chemical or biological agent of interest are believed to have the same mode of action with respect to the biological reaction; and wherein the reference chemical or biological agent is exposed at a concentration in vitro equivalent to the exposure levels experienced in vivo;
(d) determining the quantitative value of the endpoints of the in vitro tests administered in (c) above for each of the individual donors in the sample at each exposure level tested; and
(e) analyzing the quantitative values of step (d) to develop a mathematical transform between the endpoint score for an individual on the in vitro assay with the reference agent, and a prediction of whether or not that individual would experience a reaction to the reference agent in vivo, wherein the individual of the population of interest is a mammal.

### DETAILED DESCRIPTION OF THE INVENTION

A method is described herein for determining threshold limits, such as dose, exposure, or concentration limits, for a chemical or biological agent to which individuals in a population of interest may be exposed. In accordance with the method, results of in vitro exposure to the agent of interest as well as results of in vitro exposure to a reference agent are combined with data obtained regarding the incidence of the in vivo effects of the reference agent. This combination is utilized to either (1) establish one or more scores for one or more endpoints of an in vitro experiment that can serve as a proxy for estimating a threshold level of a beneficial or detrimental effect, or (2) estimate or predict, a priori, the potential incidence of a beneficial or adverse biological reaction by individuals in a population of interest when exposed in vivo to the chemical or biological agent of interest.

### Definitions

For purposes of this application, the following terms shall have the definitions described below:

In vitro, as used herein, includes, but is not limited to, assays or tests using any construct outside of the body or bodies of the donor involving cells that are maintained in a live state, and that were derived from cells originating in one or more members of a population of interest or a population postulated as being analogous to that population of interest. Thus, the term specifically includes, but is not limited to: (1) any traditionally defined in vitro tests involving individual live cells or aggregations of live cells, whether they be primary cells, stem cells, or cells derived from those cells, such as differentiated cells; (2) any tests involving tissue or organ constructs created wholly or partially from such cells, including those constructs that involve synthetic or engineered materials; (3) any tests conducted outside of the body on tissues from the donor that are maintained in a live state, whether those tissues have been removed directly from the donor, or created by the aggregation or biological development of cells included in section 1 above; and (4) any construct involving multiple cell types or tissue types wherein the cells or tissues are maintained in a live state, and some or all tissues or cells directly or indirectly communicate with each other, or directly or indirectly pass nutrients or chemical signals to each other (e.g., in silico experiments).

Threshold or Threshold Limit is defined herein as an amount or level specified as being of interest. “Threshold” may refer to single thresholds, or multiple thresholds (such as the levels that correspond to a reaction by 20 percent, 40 percent or 60 percent of the population). For example, a threshold may be the minimum or maximum point such as dose, exposure, or concentration at which a biological reaction of interest takes place or reaches an intensity where it becomes relevant to the biological reaction of interest.

Agent, as used herein, may be a single substance or biological entity (such as a pharmaceutical compound, or any other medicine or vaccine, or a chemical, virus or bacteria), or something formed by any combination of substances or biological entities. For example, an agent could consist of a pharmaceutical compound, a mixture of compounds (such as those employed in drug-drug interaction testing), or a mixture of chemical and biological agents (such as two or more infectious agents, or an infectious agent and a chemical compound). Further, the term “agent”, as it is used herein, is defined to encompass protocols in which the multiple agents or compounds referred to in the previous sentence are added or withdrawn at different times—such as in a drug-drug interaction study wherein the protocol calls for measurements to be taken after the addition of the first compound, then after the addition of a second compound, and then for further measurements to be taken thereafter.

Agent of Interest, as used herein, refers to one or more agents (such as a pharmaceutical compound, etc.) for which the method is intended to provide an estimate of the incidence within a population of a specified effect, if or when that population is exposed to that agent. The agent of interest need not be specified in advance of any experiments conducted on a reference agent, or of the development of the relationship and mathematical transform (see definition, below) of the effects of the reference agent (see definition, below) in vitro and in vivo.

Reference Agent, as used herein, refers to any agent (such as a pharmaceutical compound, etc.) that is chosen due to its postulated similarity of reactions to the agent of interest, and for which the following are, or can be made, available: (1) data on the incidence of reactions by the population of interest when exposed to the agent in vivo; and (2) results of one or more assays that are postulated to be predictive of that incidence and that can be operationalized on the reference agent as well as the agent of interest.

Incidence, as used herein with respect to in vivo assays, refers to the portion of a sample or population that did, or is predicted to, experience a reaction (or a reaction above the threshold limit) when exposed to an agent. With respect to in vivo assays, the term incidence is used herein to refer either to the portion of the total sample, or to the collection of end point scores experienced by all the members of the sample when exposed to the agent, wherein a judgment is rendered regarding what portion of those scores represents scores that indicate a reaction of sufficient magnitude to be salient in the present experiment (i.e., that a threshold limit is surpassed).

Mathematical Transform, as used herein, refers to any mathematically-based or graphically-based algorithm or translation that produces a repeatable conversion from a quantified value on one or more in vitro measures to a quantified value (including a binary measure, such as “effect present versus effect absent”) on an in vivo measure. As used herein, the term includes, but is not limited to, linear relationships, non-linear relationships, outputs of linear or non-linear regression models, clustering, self-organizing maps or related methods, or any other arithmetic conversion formula. A mathematical transform may combine multiple inputs from multiple tests in any way that is either jointly predictive, or in serial or parallel form. Further, both the inputs and outputs of the mathematical transform are not limited to point values, but may include ranges, estimates, or directional relationships. An example of a mathematical transform (referred to herein as the “Reaction Threshold Prediction Model”) is provided in one of the embodiments described below.

Sample, as used herein, may include a random sample, a representative sample, a stratified sample, or any other subset of a population that is created for the purpose of providing insight about the population as a whole, or about any designated sub-portion of the population as a whole. While the method described herein may adopt the descriptive shortcut of implying that the portion of a sample that exhibits a characteristic (such as a reaction to a compound) is the same portion as would be exhibited by the population as a whole, it should be understood that the scaling-up techniques from incidence in the sample to incidence in the population are applied as appropriate to the choice of sampling technique. These techniques are well known to those skilled in the art.

Individual, as used herein, is defined as an identifier at the level of a specific person, or a specific member of a cohort of animals in the case of the invention being applied to other species of mammals, who was the original source of the cells or tissues used in the method, and/or for whom results of assays are directly measured.

It is further noted that, as used herein, the words “a” and “an” mean one or more and that the singular forms “a”, “an” and “the” include plural referents unless expressly and unequivocally limited to one referent.

### Steps and Factors

Described herein is a method for estimating or predicting the potential incidence of biological reactions by an individual in a population of interest when exposed to an agent of interest in vivo. Preferably, the population of interest is a mammalian population, preferably the individual is a human and the agent of interest is a chemical or biological agent of interest. The estimation or prediction is achieved using a combination of (1) in vitro experiments on the agent of interest and in vitro experiments on a reference agent; and (2) data obtained regarding the incidence of in vivo effects of exposure to the reference agent.

The method involves two sets of steps: (1) develop a mathematical transform that relates the pattern of scores on endpoints in one or more in vitro assays to the incidence of reactions in vivo via analysis of a reference agent for which both the in vivo and in vitro results are known; and (2) apply that mathematical transform to the end point scores resulting from those same in vitro assays when the agent of interest is tested, to develop an estimate of the likely in vivo results were the agent of interest to be encountered in vivo. The first set of steps may be pursued alone to add to the future usefulness of the assay, or the results of the first set of steps may be combined with the second set of steps to provide estimates for agents of interest.

At least three categories of factors, causally rather than simply correlative, influence the accuracy of the resulting estimate:

A first category of factors relates to the degree of similarity between the causation of effects in vivo and the endpoints measured by the in vitro assay. These similarities are greater when the cells used in the in vitro experiments are robust models of their in vivo counterparts; the effects seen in vivo are the causal result of impacts on the precise tissue constructs analyzed in the in vitro assay (e.g., the result of cellular-level damage, rather than organ-level or system-level damage, when the in vitro assay is cellular based); and the in vitro assay measures the same modes of action that cause the in vivo effect, which in turn assumes all the modes of action are known.

A second category of factors relates to the similarity among, and representativeness of, the population samples used in the sources of data input to the mathematical transform, as well as the similarity between the population from which the in vivo effects were measured, in the case of the reference agent, and the population for which the effects are being estimated, in the case of the agent of interest. For example, in a drug toxicity application, the input sources might consist of the clinical trial patient sample that produced the in vivo results for the reference compounds; the donor sample from which cells were originated for use in the in vitro assay of the reference compounds; and the donor sample from which cells were originated for use in the in vitro assay of the compound of interest. Greater similarity of these three samples, with each other within the population for whom the compound of interest may ultimately be intended, contributes to better accuracy. A number of aspects of similarity should be considered, including demographic phenotypes of donors and the presence or absence of health conditions of donors, which might affect or contribute to the end point scores of the assays.

A third category of factors relates to the similarities between the reference agent and the agents of interest. Any important similarities or differences between the reference agent and the agents of interest in pharmacokinetics, in the number of modes of action, in the nature of those modes of action (for example, which modes of action remain unmeasured between the reference agent and the agent of interest), and/or in the rate of change of reaction along one or more endpoints as levels of exposure change, are key in this regard.

Various embodiments of the method provided herein explicitly control for different combinations of the above-described factors. Further, it is anticipated that the level of control and consistency of these factors will improve over time as the number of reference agents tested increase, as the variety and quality of the underlying assays increase, as researchers produce or gain access to more complete databases on the effects of reference agents that have been experienced in vivo, etc.

The accuracy of the quantitative relationships embodied in the mathematical transforms may exceed the level that might be expected through strictly causal modeling of the linkages described above, even in embodiments that do not attempt to control many of the three categories of factors just described. This improved accuracy occurs because many pharmacodynamics effects—even if unmeasured by the particular assays employed—are likely correlated to each other. For example, an agent may harm several types of cells in roughly equal proportions, and each of those harms might contribute to an in vivo reaction. Even if an assay only measures damage to one type of cell, the total in vivo effect may still be tightly correlated with the assay's end point score. Alternatively, a mode of action that involves the breakdown of communication between two or more cell types or organs (such as the nervous system's signaling to the heart) can be correlated with an assay measuring underlying damage to only one of the cell types or organs involved, as damage to either cell type or organ would interfere with effective communication between them.

For this reason, the present method includes a wide variety of embodiments, many of which will vary not only in purpose, but also in underlying design elements that exert control over the various factors described above.

### Conditions

The method can be applied under any circumstance in which the following conditions are present: (1) the in vivo nature of the biological reaction of interest has previously been defined; (2) reactions of that nature have been observed, and the incidence of those reactions has been recorded in a population that was analogous to the population of interest, when that analogous population was exposed in vivo to at least one reference agent (chemical or biological) whose mode of action is believed a priori to be sufficiently similar to the agent of interest to potentially produce a usefully accurate mathematical transform; (3) at least one in vitro assay has been identified that the researcher postulates is causally or coincidentally correlated with the mode of action identified in (2) above; (4) the researcher can physically obtain both a reference agent for which the in vivo incidence was measured above and the agent of interest; and (5) the in vitro assay can successfully be operationalized on both the reference agents and the agent of interest.

Optionally, an additional condition may affect the method provided herein, one which can be most easily described in the context of pharmaceutical compounds. In this context, a compound varies from other compounds not only in its impact on individual humans (i.e., pharmacodynamics), but also in how the bodies of different individual humans absorb and modify (or metabolize) the compound before, during, and after the compound contacts specific cells it might beneficially or adversely affect. In the context of pharmaceuticals, this is called pharmacokinetics: in the context of any agent, the more general term “agent kinetics” will be used herein.

A researcher's ability to use the combined in vitro and in vivo data from one or more reference agents to predict in vivo effects of an agent of interest involves explicitly or implicitly addressing the similarity or dissimilarity of agent kinetics across the agents. There are three strategies for addressing agent kinetics. First, in cases where the choice of reference agent is quite limited (e.g., due to a limited number of agents on which both the in vivo data is available and an analogous in vitro assay has been developed), the researcher may have no choice but to ignore differences in agent kinetics in the development of the model itself, and rely on external analysis of the similarities or differences in agent kinetics to scale up or down the dose concentrations or exposures of the agents to correct for these differences, or, less preferably, simply judge how accurate or inaccurate the resulting predictions can be. Second, as the number of potentially suitable reference agents increases, the researcher can control for agent kinetics differences by deselecting any potential reference agents whose agent kinetics is too dissimilar to the agent of interest's agent kinetics. Third, as the number of potentially suitable reference agents increases further, the researcher may be able to build a mathematical transform that embeds the effects of differences in agent kinetics into its calculations.

### Method of Predicting the Incidence or Degree of an In Vivo Reaction

The method of predicting the incidence or degree of an in vivo reaction, as provided, herein contains two steps, each further including multiple sub-steps.
1. Establish the mathematical relationships between incidence in vivo and endpoint scores for in vivo experiments.
   a. Determine biological reaction of interest in vivo, and in vitro assays to be used.

The reaction of interest in vivo is identified for which a prediction of the incidence for the agent of interest is desired. While it may be easy to identify the general category of reaction (e.g., cardiotoxicity during a drug development program), it will likely be necessary to define the reaction more specifically in order to meet two separate constraints. First, the reaction must be one for which data can be found regarding the incidence of that reaction experienced in vivo. For example, in drug toxicity testing, this data is most likely to be found from Phase II or III clinical trials of a reference compound. Depending on the databases to which there is access, as well as the availability of reference agents themselves, the specific definition of the reaction may require adjustment. For example, when using the label warnings of approved drugs as the source of information on incidence in vivo, the researcher is limited to a highly idiosyncratic set of information for each drug regarding the nature and incidence of adverse events. This information may or may not match the definitions of events in the same class of toxicity reported in the warning labels of other reference compounds. Second, there must be available an in vitro assay that can be postulated as being predictive (due to causality or correlation, or a combination of the two) of the incidence of the reaction in vivo. Again, this may require a compromise on the specific definition of the reaction of interest.
b. Select and prepare sample of cells, tissues, etc. to be tested in vitro.

The cells, tissues, etc. required by the chosen in vitro assay are obtained from a sample of donors who collectively represent the population of interest (i.e., the population for whom the incidence of reaction is to be predicted). In this regard, the term “obtained” includes directly extracting or gathering the cells or tissues from the donor via any method and/or developing the type and number of cells or tissues required via culturing, growing, differentiating and/or manipulating such cells. Methods for defining the sample and the requirements for obtaining and preparing the required cells or tissues are described in PCT Application No. PCT/US14/45499, entitled “Methods for Predicting Responses to Chemical or Biologic Substances” and PCT Application No. PCT/US14/53819, entitled “Methods For Genetically Diversified Stimulus-Response Based Gene Association Studies”, both of which are incorporated by reference herein. Exemplary cells include, but are not limited to, stem cells or cells derived, induced (induced pluripotent stem cells) or differentiated therefrom. It is strongly preferred, but not required, for the cells or tissues that will be tested by both the reference agent and the agent of interest to be drawn from the identical sample of donors. The number of donors is preferably at least 10, at least 30 or at least 100 donors.
c. Conduct in vitro assay on reference agents.

Necessary quantities are obtained of the one or more reference agents for which the researcher was able to obtain the in vivo reaction incidences referred to in Step 1a above. The selected in vitro assay is then conducted on a portion of the cells or tissues obtained in Step 1b above, at the dose or exposure equivalents to the in vivo doses or exposures for which the incidence data was previously obtained using methods well known by those skilled in the art to equilibrate the in vitro concentration to the in vivo exposures or concentrations. The end point scores of the in vitro tests are then recorded for each donor.
d. Rank donors.

For each time the assay is conducted on a reference compound (wherein a single repetition is the administration to all donors of a single dose of a single compound), the donors are then ranked in order of ascending or descending end point scores. Such ranking is in ascending order when higher scores are believed to be associated with stronger reactions, and descending order when lower scores are believed to be associated with stronger reactions.
e. Identify sufficient set of threshold scores.

Sub-step 1c and 1d are repeated for additional exposures (for which in vivo information on incidence is available) and/or for additional reference agents (potentially at multiple doses as well, if information is available) until a sufficient set of threshold scores is identified. The resulting threshold scores are then examined collectively to determine whether they are appropriately distributed to form the basis of useful estimates or predictions.
f. Develop mathematical transform.

Using any appropriate mathematically-based or graphically-based algorithm or translation (as described in the definitions provided above), the incidence and/or strength of reactions within the individuals tested in vitro are related to the incidence of reactions seen in vivo under analogous conditions. The development of the mathematical transform may take place serially, in which certain exposures and/or agents are used as a training set to develop an initial version of the transform, which is then tested for predictive accuracy against other exposures and agents. Following these tests, the mathematical transform can then be modified to incorporate additional data and findings.
2. Combine mathematical relationships with the of assays of compounds of interest to estimate incidence of reactions in a population.
a. Conduct in vitro assay.

The in vitro assay referenced in steps 1c, 1d, and 1e above is conducted on the compound of interest at a dose of interest, using cells or tissues derived from a sample of donors representative of a population of interest. Preferably, the cells or tissues will include some or all of the cells or tissues obtained in step 1b above, or cells or tissues derived therefrom. The end point scores are noted for each of the donors and the donors are arrayed according to their end point scores, as in 1d above.
b. Estimate/predict incidence of reactions that would occur if population of interest were to be exposed to agent of interest.

The estimate/prediction is created by combining the outputs of steps 1f and 2a in ways that are well known to those skilled in the art. In some embodiments, one endpoint (even with multiple reference agents and multiple exposures) will not be sufficient to establish a robust mathematical transform between the results of in vitro assays on a training set of reference agents and in vivo results of that same training set of reference agents in order to use the mathematical transform to confidently posit the relationship between the in vitro results for an agent of interest and predicted in vivo results of that same agent of interest.

Therefore, where multiple modes of action are in play, the researcher may utilize multiple end points from the same assay or multiple in vitro assays to obtain a variety of endpoints that correspond directly or indirectly with the various modes of action involved in the in vivo experience. All of these may then be combined in the mathematical transform.

### Exemplary Embodiments of the Described Methods

As mentioned throughout this description, the method provided herein can be manifested in a wide variety of embodiments, where the variation can take place along multiple dimensions. The following embodiments are not intended to be exhaustive, but are intended only to illustrate certain aspects of this variety.

One embodiment is a simple development and application of a “Reaction Threshold Prediction Model” referred to above. In this embodiment: (1) data describing the impact of reference agents in vivo is limited to incidence only (i.e., the data specifies only the portion of the population exposed to the agent that experienced any reaction; it does not specify the degree of reaction of individuals); and (2) only one in vitro assay is considered relevant, and that assay only reports one end point.

In this embodiment, it is assumed that an individual's reaction in vivo to a particular exposure to a reference agent is tightly related to the end point score that the same individual's cells/tissues would register in the in vitro assay when exposed to the analogous concentration of the same agent. That is, using a drug development toxicity example, it is assumed that the in vitro assay measures damage to the cells or tissues being challenged by the compound. It is further assumed that there is relatively little variation from human to human in the degree of damage that a person can sustain in vivo to those same types of cells or tissues before manifesting a toxicity reaction of interest. Therefore, in this embodiment, “damage” is measured by the percentage of cells that are binarily classified as “harmed”. No continuous measurement of the degree of harm is taken at the level of a single cell. Importantly, it is assumed nearly the opposite with respect to the dose concentration that would be required to cause that level of damage. It is assumed that there is great variation individual to individual as to the level of damage that would be caused by one particular dose concentration.

Therefore, it is postulated that there is a threshold limit (or, more realistically, a “zone”) of the endpoint in vitro that divides those who would have a reaction in vivo from those who would not. The threshold is determined by finding the endpoint level at which the portion of the sample exceeding that threshold is the same as the incidence portion of the population exposed to the same reference compound in vivo. Should a sufficiently consistent threshold be found when the process is repeated across multiple exposures of a single reference agent (or, preferably, across multiple exposures of multiple reference agents), then it may be postulated that the end point threshold of the in vitro assay is a sufficiently reliable indicator of the onset of the reaction for the agent of interest. That portion is then used to scale up to a projection for the population as a whole.

For example, if, in one pairing of an in vitro test of a given reference agent to its corresponding in vivo test, 17% of the patients in the in vivo test experienced an adverse reaction, one assumes that 17% of the donors whose cells or tissues were used in the in vitro test would have experienced a similar adverse reaction. The end point score on the in vitro assay can then be determined that would be required to distinguish between the 17% of patients who are assumed to be vulnerable from the 83% of patients who are assumed to be not vulnerable. This score becomes the threshold limit. That same in vitro assay is then administered using a chosen concentration of the agent of interest, and the portion of the sample whose score exceeds that threshold limit is computed.

In another embodiment, data may be available on multiple aspects of the reactions in vivo, such as the degree of reaction experienced by sub-portions of the population that have been exposed to the reference agent. In this embodiment, more sophisticated techniques may be used in developing the mathematical transform to estimate a relationship between various endpoint scores from the in vitro assay and the probability of experiencing a reaction, or a reaction of a certain degree, in vivo. As above, those scores are then assumed to apply to the agent of interest.

In another embodiment, multiple assays are relevant and available, or multiple endpoints may be read from the same assay. In such a case, the mathematical transform may involve any technique such as linear regression, clustering, self-organizing mapping, etc., wherein the impacts of multiple endpoints are combined to form a single predictive output.

In another embodiment, it may be possible to administer the in vitro assays to cells or tissues derived from the specific individuals who experienced the in vivo exposures to reference agents and who subsequently reacted (or did not react) to their exposure. In this embodiment, the relationship between in vitro endpoint scores and in vivo outcomes can be mathematically explored in depth and new mathematical transforms developed. The specific translations of these relationships into ones to use with the agent of interest will depend on whether the population of interest, in connection with the agent of interest, is the one originally exposed to the reference compound, or one for which that population is simply a proxy.

The method provided herein may be better understood by reference to the following non-limiting examples.

### EXAMPLES

### Example 1

### Determining the Potential of a Compound For Patient Use

A pharmaceutical compound, Compound X, is being studied at an early stage of the drug development process. Compound X is chemically similar to, but not identical to, doxorubicin, which is currently marketed as a potent anti-cancer medication, but that is known to have significant cardiotoxic side effects when administered in therapeutic doses. Doxorubicin's toxicity is severe enough to act as an impediment to the usage of the drug. In fact, its toxicity is severe enough that the FDA would be unlikely to approve other compounds with comparable toxic severity unless they, like doxorubicin, were prescribed only under circumstances where there were few or no acceptable alternatives.

A researcher believes that Compound X (at its projected therapeutic dose) would produce significantly lower cardiotoxic effects than doxorubicin produced in clinical trials. The researcher establishes the criterion that, if Compound X, at therapeutic doses, is likely to have the same or a higher incidence of the type of cardiotoxic effects as doxorubicin, the development of Compound X will be discontinued.

The clinical incidence of significant cardiotoxicity events of doxorubicin at doses of 500 mg and 600 mg are catalogued, by obtaining relevant information from the warning label mandated by the Federal Drug Administration.

An in vitro assay is then identified whose endpoint—referred to herein as Endpoint A—includes one that is causally associated with one of the cardiotoxic effects of doxorubicin: cardiomyopathy. Concentrations of doxorubicin as prescribed by the in vitro assay's protocol are prepared in a concentration that corresponds to doses of 500 mg and 600 mg administered in vivo.

In vitro tests are conducted at these concentrations on a representative sample of cardiomyocytes derived from stem cells (the cardiomyocytes are differentiated induced pluripotent stem cells) originating from a sample of 100 donors, and then the donors are arrayed in order of ascending scores on Endpoint A.

The “Reaction Threshold Prediction Model” is selected as the mathematical transform in his procedure. It is noted from the doxorubicin clinical cardiotoxicity data mentioned above that 12% of the clinical trial participants experienced cardiomyopathy when treated at the 500 mg dose level. Using the in vitro test results above, the Endpoint A score is determined that is associated with the 88^{th} Percentile member of the in vitro cohort of 100 donors (88 representing 100% minus the 12% of the sample in vivo who experienced cardiomyopathy following treatment with a 500 mg dose). The Endpoint A score reads 70 units.

Next, the in vitro assay of doxorubicin is repeated, with a concentration corresponding to a 600 mg dose administered in vivo, on a representative sample of cardiomyocytes derived from stem cells originating from a sample of 100 donors, and then the donors are arrayed in order of ascending scores on Endpoint A. This time, the Endpoint A score is detected for the donor at the 76^{th} Percentile (76 representing 100% minus the 23% of the sample in vivo who experienced cardiomyopathy following treatment with a 600 mg dose). This time, the Endpoint A score reads 65 units.

It is therefore postulated that, in the case of doxorubicin, an Endpoint A score between 65 and 70 approximates a threshold of impact that leads to incidences of cardiomyopathy in the range of 10 to 25 percent. It is further hypothesized that the same threshold of impact would apply to Compound X. The doses of Compound X will not be the same as the doses of doxorubicin used to discover the presumed toxicity threshold of 65-70, but it is believed that this does not matter—rather, what matters at any concentration of interest is the portion of the sample who experience an Endpoint A score above the threshold of 65-70.

Next, the in vitro assays are conducted on cardiomyocytes from the same sample of 100 donors, this time under challenge by Compound X, administered at concentrations of interest (i.e., ones that correspond to the doses required in vivo to be effective against Compound X's chosen therapeutic target).

It is determined that, at the concentrations of interest, the portion of the sample experiencing an Endpoint A score above 65 units is 24%, and the portion experiencing a score above 70 units is 17%. It is noted that the portion of the sample “adversely affected” by Compound X is within the range of the portion of the sample “adversely affected” by doxorubicin. Therefore, it is estimated that that the portion of a population treated by Compound X who would experience cardiomyopathy in vivo would be likely be in the same general range as the incidence of clinically experienced cardiomyopathy when treated by doxorubicin. Therefore, the development of Compound X is discontinued.

### Example 2

### Developing a Threshold For Estimating the Incidence of Cardiotoxicity For Novel Compounds When They Are Evaluated by a Specified In Vitro Assay

A researcher desires to create a generalized model to use in conjunction with a new assay in order to estimate the potential incidence of cardiotoxicity in humans of a set of novel compounds. In particular, the researcher is concerned with oxidative stress leading to hypertrophic cardiomyopathy. The in vitro assay measures reductions in the cell viability of cardiomyocytes through measuring changes in cell membrane integrity.

However, prior to the method described herein, no method exists to translate the numerical output from the measured endpoint of the assay (i.e., percent of cardiomyocytes whose cell membrane integrity has been damaged) into an estimate of the likelihood of hypertrophic cardiomyopathy in vivo.

Therefore, the researcher examines the literature surrounding the cardiomyopathy of a widely used drug—Compound B—and discovers a key study in which the reported incidence of cardiomyopathy inpatients was 18 percent when Compound B was administered at the 200 mg dose, and the incidence was 36 percent when Compound B was administered at the 400 mg dose.

The researcher decides to use Compound B at the in vitro dose's equivalent to 200 and 400 mg as the first reference compound for establishing an estimate of the endpoint score on the assay that corresponds to a threshold for the onset of cardiomyopathy.

The researcher conducts the assay specified above (i.e., cell membrane integrity) on iPSC-derived cardiomyocytes that have been prepared from cells from a sample of 24 donors who constitute a representative sample of the population of interest, wherein those cardiomyocytes are challenged by doses of Compound B at in vitro doses equivalent to 200 and 400 mg. The researcher then rank orders the endpoint scores (from lowest to highest) in order to determine scores corresponding to the 82^{nd} and 64^{th} percentiles of the distributions respectively (being the converse of the 18 and 36 percent incidence rates).

The endpoint scores at the specified percentiles are 40 and 45 (percent of cells experiencing a loss of cell membrane integrity) respectively. Accordingly, the researcher preliminarily concludes that the threshold score on this particular assay corresponding to the potential for high likelihood of hypertrophic cardiomyopathy is in the range of 40 to 45 percent.

The researcher then conducts similar assays on the same cells using other reference compounds whose in vivo incidence of hypertrophic cardiomyopathy has been documented. The results of these experiments produce estimates for the threshold endpoint score in the range of 35 to 45.

The researcher adopts the hypothesis that the appropriate endpoint score for this assay to use for estimating what portion of the population would likely experience hypertrophic cardiomyopathy when treated with a novel compound would be 35 to 45 (percent of cells experiencing a loss of cell membrane integrity). The portion of the sample that scores in or above this range would correspond to the estimated portion that would be at high risk for hypertrophic cardiomyopathy when treated at the corresponding in vivo dose.

The researcher then arranges to conduct the same assay using several additional reference compounds (for which the incidence of in vivo cardiomyopathy has been documented) on a blinded basis. In each case, the researcher uses the distribution of end point scores from the in vitro assay and the mathematical transforms developed above in this example to estimate the portion of the population experiencing cardiomyopathy in vivo. When the compounds are unblended, the researcher analyses the accuracy of his estimates/predictions against the actual in vivo data, using statistical techniques well known in the field. The researcher concludes that the in vitro test provides a usefully accurate prediction. The results are then published and shared with the industry and regulators in a process to build confidence within the industry for using the test and mathematical transform in assisting stage-gating decisions during the drug development process.

Although the foregoing has been described in some detail by way of illustration and example for purposes of clarity and understanding, one of skill in the art will appreciate that certain changes and modifications can be practiced within the scope of the appended claims. In addition, each reference provided herein is incorporated by reference in its entirety to the same extent as if each reference was individually incorporated by reference.

1. A method for estimating or predicting the incidence or degree of a specified biological reaction in an individual in a population of interest when exposed to a chemical or biological agent of interest in vivo, comprising:
(a) determining the incidence or degree of the biological reaction in a sample of the population of interest, wherein the sample comprises cells or tissues from at least 10 individual donors of the population of interest, or an analogous population, when exposed to at least one reference agent in vivo;
(b) determining the incidence or degree of the biological reaction in a sample of the population of interest, wherein the sample comprises cells or tissues from at least 10 individual donors of the population of interest, or an analogous population, when exposed to the at least one reference agent in vitro;
(c) determining the incidence or degree of the biological reaction of individuals in the population of interest when exposed to the chemical or biological agent of interest in at least one in vitro assay; and
(d) estimating or predicting the incidence or degree of the biological reaction by an individual in the population of interest based on the incidence or degree of the biological reactions in step (a), wherein the individual of the population of interest is a mammal.

2. The method of claim 1, wherein the mammal is human.

3. The method of claim 1, wherein the number of donors in step (b) is at least 30.

4. The method of claim 1, wherein the number of donors in step (b) is at least 100.

5. A method for establishing a threshold endpoint score for an in vitro assay to produce an estimate of the incidence in a population of interest of a related effect in vivo, comprising:
(a) selecting one or more in vitro tests for which an endpoint measured by those tests may exhibit a mathematical transform with the incidence of the biological reaction in vivo;
(b) identifying at least 10 individual donors as a sample of the population of interest, and obtaining separately for each donor, a cell, tissue, organ, aggregates thereof or cells, tissues, or organs derived therefrom for the selected in vitro test;
(c) administering the selected in vitro test on each donor of each sample by exposure to a reference chemical or biological agent, wherein the incidence of the biological reaction has been determined for the in vivo exposure of the sample population of interest or an analogous population to the reference chemical or biological agent; wherein the reference chemical or biological agent and the chemical or biological agent of interest are believed to have the same mode of action with respect to the biological reaction; and wherein the reference chemical or biological agent is exposed at a concentration in vitro equivalent to the exposure levels experienced in vivo;
(d) determining the quantitative value of the endpoints of the in vitro tests administered in (c) above for each of the individual donors in the sample at each exposure level tested; and
(e) analyzing the quantitative values of step (d) to develop a mathematical transform between the endpoint score for an individual on the in vitro assay with the reference agent, and a prediction of whether or not that individual would experience a reaction to the reference agent in vivo, wherein the individual of the population of interest is a mammal.

6. The method of claim 5, wherein the mammal is human.

7. The method of claim 5, wherein the number of donors in step (b) is at least 30.

8. The method of claim 5, wherein the number of donors in step (b) is at least 100.

9. The method of claim 5, wherein the mathematical transform is the reaction threshold prediction model.

10. A method for estimating or predicting the incidence or degree of a specified biological reaction, among individuals in a population of interest of mammals when exposed to a chemical or biological agent of interest in vivo, comprising:
(a) selecting one or more in vitro tests for which an endpoint or endpoints measured by those tests may exhibit a mathematical transform with the incidence of the biological reaction in vivo;
(b) identifying at least 10 individual donors as a sample of the population of interest, and obtaining separately for each donor, a cell, tissue, organ, aggregates thereof or cells, tissues, or organs derived therefrom for the selected in vitro test;
(c) administering the selected in vitro test on each donor of that sample using a reference chemical or biological agent, wherein the incidence of the biological reaction has been determined for the in vivo exposure of the sample population of interest or an analogous population to the reference chemical or biological agent; wherein the reference chemical or biological agent and the chemical or biological agent of interest have the same mode of action with respect to the biological reaction; and wherein the reference chemical or biological agent is used at a concentration in vitro equivalent to the exposure levels experienced in vivo;
(d) determining the quantitative value of the endpoints of the in vitro tests administered in (c) above for each of the individual donors in the sample at each exposure level tested;
(e) analyzing the quantitative values of step (d) to develop a mathematical transform between the endpoint score for an individual on the in vitro assay and the reference agent, and a prediction of whether or not that individual would experience a reaction to the reference agent in vivo;
(f) conducting the in vitro tests on the cells, tissues, or organs of each of the donors using the chemical or biological agent of interest at various concentrations to obtain scores for each donor on the endpoints of interest;
(g) calculating the portion of the sample donors whose scores on each endpoint when exposed to the agent of interest lie beyond the threshold limit; and
(h) using the results of the step (g) to estimate the potential incidence of the biological reaction by the individual in the population of interest.

11. The method of claim 10, wherein step (e) comprises determining, for each exposure level of each endpoint for each agent separately, the threshold score that would divide the in vitro sample population into donors having a score higher than the threshold score versus donors having a score lower than the threshold score in the same proportion as the incidence of the reaction of interest found in vivo to obtain a data set of observations, wherein, for each observation, the agent, the threshold score, and an incidence of toxicity are included.

12. The method of claim 10, wherein the mammals are human.

13. The method of claim 10, wherein the number of donors in step (b) is at least 30.

14. The method of claim 10, wherein the number of donors in step (b) is at least 100.

15. The method of claim 10, wherein the mathematical transform is the reaction threshold prediction model.
